SEQUENCE LISTING (12 sequences)

SEQ ID NO: 1 - DNA, 11 bp, Artificial Sequence
  Synthetic Oligonucleotide
ctaggcctgtc

SEQ ID NO: 2 - DNA, 11 bp, Artificial Sequence
  Synthetic Oligonucleotide
gacaggcctag

SEQ ID NO: 3 - DNA, 11 bp, Artificial Sequence
  Synthetic Oligonucleotide
ctaggcctgtc

SEQ ID NO: 4 - DNA, 11 bp, Artificial Sequence
  Synthetic Oligonucleotide
gacaggcctag

SEQ ID NO: 5 - DNA, 11 bp, Artificial Sequence
  Synthetic Oligonucleotide
ctaggcctgtc

SEQ ID NO: 6 - DNA, 11 bp, Artificial Sequence
  Synthetic Oligonucleotide
gacaggcctag

SEQ ID NO: 7 - DNA, 11 bp, Artificial Sequence
  Synthetic Oligonucleotide
ctaggcctgtc

SEQ ID NO: 8 - DNA, 11 bp, Artificial Sequence
  Synthetic Oligonucleotide
gacaggactag

SEQ ID NO: 9 - DNA, 11 bp, Artificial Sequence
  Synthetic Oligonucleotide
ctaggcctgtc

SEQ ID NO: 10 - DNA, 11 bp, Artificial Sequence
  Synthetic Oligonucleotide
gacaggactag

SEQ ID NO: 11 - DNA, 11 bp, Artificial Sequence
  Synthetic Oligonucleotide
ctaggcctgtc

SEQ ID NO: 12 - DNA, 11 bp, Artificial Sequence
  Synthetic Oligonucleotide
gacaggactag

(19) United States
(12) Patent Application Publication
(10) Pub. No.: US 2010/0137154 A1
(43) Pub. Date: Jun. 3, 2010

(54) GENOME ANALYSIS USING A METHYLTRANSFERASE

(76) Inventors: Robert A. Ach, San Francisco, CA (US); Brian J. Peter, Los Altos, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 12/325,562

(22) Filed: Dec. 1, 2008

(51) Int. Cl. C40B 30/04; C12Q 1/68

(52) U.S. Cl. 506/9; 435/6

(57) ABSTRACT

A method of genome analysis is provided. In certain embodiments, the method may comprise: labeling the test genome using a first site-specific methyltransferase to produce a labeled test genome comprising a label; and analyzing the labeled test genome to determine if the test genome comprises a sequence alteration relative to a reference sequence. In certain embodiments, the method may comprise: evaluating binding of the labeled test genome to an array of probes, or observing a pattern of labeling along the labeled test genome.

### BACKGROUND

Despite widespread use of microarrays and sequencing for genetic analysis, there remains a need for new methods to analyze DNA. Particularly, there are limited methods for identifying or analyzing large, unbroken stretches of genomic DNA. A method which provides higher resolution than karyotyping or FISH, but is easier to implement than Fiber-FISH, could potentially identify inversion and balanced translocations that are difficult to detect by current methods.

DNA methyltransferases are a class of enzymes that attach methyl groups to specific DNA bases. Bacteria have a class of DNA methyltransferases the specifically recognize certain restriction enzyme sites and can attach methyl groups to specific bases within those sites. Methyltransferases with specific recognition sites are commercially available. Bacteria utilize this system to protect their own chromosomes from being cut by the restriction enzymes they produce. In mammals, DNA methyltransferases recognize the CpG dinucleotide, and will methylate this sequence when appropriate, as a means to regulate gene expression.

This disclosure relates in part to a method of genome analysis using a methyltransferase.

### SUMMARY

A method of genome analysis is provided. In certain embodiments, the method may comprise: labeling the test genome using a first site-specific methyltransferase to produce a labeled test genome comprising a label; and analyzing the labeled test genome to determine if the test genome comprises a sequence alteration relative to a reference sequence. In certain embodiments, the method may comprise: evaluating binding of the labeled test genome to an array of probes, or observing a pattern of labeling along the labeled test genome.

### BRIEF DESCRIPTION OF THE FIGURES

FIG. 1 schematically illustrates an embodiment of the method described herein.

FIG. 2 schematically illustrates certain features of some embodiments of the method described herein.

FIG. 3 schematically illustrates certain features of another embodiment of the method described herein.

### DEFINITIONS

The term “sample”, as used herein, relates to a material or mixture of materials, typically, although not necessarily, in liquid form, containing one or more analytes of interest.

The term “genome”, as used herein, relates to a material or mixture of materials, containing genetic material from an organism. The term “genomic DNA” as used herein refers to deoxyribonucleic acids that are obtained from an organism. The terms “genome” and “genomic DNA” encompass genetic material that may have undergone amplification, purification, or fragmentation. The term “test genome,” as used herein refers to genomic DNA that is of interest in a study.

The term “reference genome”, as used herein, refers to a sample comprising genomic DNA to which a test sample may be compared. In certain cases, reference genome contains regions of known sequence information, e.g., an SNP.

The term “nucleotide” is intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the term “nucleotide” includes those moieties that contain hapten or fluorescent labels and may contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, are functionalized as ethers, amines, or the likes.

The term “nucleic acid” and “polynucleotide” are used interchangeably herein to describe a polymer of any length, e.g., greater than about 2 bases, greater than about 10 bases, greater than about 100 bases, greater than about 500 bases, greater than 1000 bases, up to about 10,000 or more bases composed of nucleotides, e.g., deoxyribonucleotides or ribonucleotides, and may be produced enzymatically or synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions. Naturally-occurring nucleotides include guanine, cytosine, adenine and thymine (G, C, A and T, respectively).

The term “oligonucleotide”, as used herein, denotes a single-stranded multimer of nucleotides from about 2 to 500 nucleotides, e.g., 2 to 200 nucleotides. Oligonucleotides may be synthetic or may be made enzymatically, and, in some embodiments, are under 10 to 50 nucleotides in length. Oligonucleotides may contain ribonucleotide monomers (i.e., may be oligoribonucleotides) or deoxyribonucleotide monomers. Oligonucleotides may be 10 to 20, 11 to 30, 31 to 40, 41 to 50, 51-60, 61 to 70, 71 to 80, 80 to 100, 100 to 150 or 150 to 200 nucleotides in length, for example.

The term “duplex” as used herein refers to a duplex formed by hybridization of two oligonucleotides containing complementary sequences, e.g. a chromosomal segment and a probe.

The term “probe”, as used herein, refers to a nucleic acid that is complementary to a nucleotide sequence of interest. In certain cases, detection of a target analyte requires hybridization of a probe to a target. In certain embodiments, a probe may be immobilized on a surface of a substrate. A “substrate” can have a variety of configurations and material, e.g., a sheet, bead, glass cover slip, or other structure. In certain embodiments, a probe may be present on a surface of a planar support, e.g., in the form of an array.

An “array” includes any two-dimensional or substantially two-dimensional (as well as a three-dimensional) arrangement of spatially or optically addressable regions bearing nucleic acids, particularly oligonucleotides or synthetic mimetics thereof, and the like. Where the arrays are arrays of nucleic acids, the nucleic acids may be adsorbed, physisorbed, chemisorbed, or covalently attached to the arrays at any point or points along the nucleic acid chain.

Any given substrate may carry one, two, four or more arrays disposed on a surface of the substrate. Depending upon the use, any or all of the arrays may be the same or different from one another and each may contain multiple spots or features. An array may contain one or more, including more than two, more than ten, more than one hundred, more than one thousand, more ten thousand features, or even more than one hundred thousand features, in an area of less than 20 cm² or even less than 10 cm², e.g., less than about 5 cm², including less than about 1 cm², less than about 1 mm², e.g., 100 μm², or even smaller. For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, 20%, 50%, 95%, 99% or 100% of the total number of features). Inter-feature areas will typically (but not essentially) be present which do not carry any nucleic acids (or other biopolymer or chemical moiety of a type of which the features are composed). Such inter-feature areas typically will be present where the arrays are formed by processes involving drop deposition of reagents but may not be present when, for example, photolithographic array fabrication processes are used. It will be appreciated though, that the inter-feature areas, when present, could be of various sizes and configurations.

Each array may cover an area of less than 200 cm², or even less than 50 cm², 5 cm², 1 cm², 0.5 cm², or 0.1 cm². In certain embodiments, the substrate carrying the one or more arrays will be shaped generally as a rectangular solid (although other shapes are possible), having a length of more than 4 mm and less than 150 mm, usually more than 4 mm and less than 80 mm, more usually less than 20 mm; a width of more than 4 mm and less than 150 mm, usually less than 80 mm and more usually less than 20 mm; and a thickness of more than 0.01 mm and less than 5.0 mm, usually more than 0.1 mm and less than 2 mm and more usually more than 0.2 mm and less than 1.5 mm, such as more than about 0.8 mm and less than about 1.2 mm.

Arrays can be fabricated using drop deposition from pulse-jets of either precursor units (such as nucleotide or amino acid monomers) in the case of in situ fabrication, or the previously obtained nucleic acid. Such methods are described in detail in, for example, the previously cited references including U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. As already mentioned, these references are incorporated herein by reference. Other drop deposition methods can be used for fabrication, as previously described herein. Also, instead of drop deposition methods, photolithographic array fabrication methods may be used. Inter-feature areas need not be present particularly when the arrays are made by photolithographic methods as described in those patents.

Arrays may also be made by distributing pre-synthesized nucleic acids linked to beads, also termed microspheres, onto a solid support. In certain embodiments, unique optical signatures are incorporated into the beads, e.g. fluorescent dyes that could be used to identify the chemical functionality on any particular bead. Since the beads are first coded with an optical signature, the array may be decoded later, such that correlation of the location of an individual site on the array with the probe at that particular site may be made after the array has been made. Such methods are described in detail in, for example, U.S. Pat. Nos. 6,355,431, 7,033,754, and 7,060,431.

An array is “addressable” when it has multiple regions of different moieties (e.g., different oligonucleotide sequences) such that a region (i.e., a “feature” or “spot” of the array) at a particular predetermined location (i.e., an “address”) on the array contains a particular sequence. Array features are typically, but need not be, separated by intervening spaces. An array is also “addressable” if the features of the array each have an optically detectable signature that identifies the moiety present at that feature.

The terms “determining”, “measuring”, “evaluating”, “assessing”, “analyzing”, and “assaying” are used interchangeably herein to refer to any form of measurement, and include determining if an element is present or not. These terms include both quantitative and/or qualitative determinations. Assessing may be relative or absolute. “Assessing the presence of” includes determining the amount of something present, as well as determining whether it is present or absent.

The term “using” has its conventional meaning, and, as such, means employing, e.g., putting into service, a method or composition to attain an end. For example, if a program is used to create a file, a program is executed to make a file, the file usually being the output of the program. In another example, if a computer file is used, it is usually accessed, read, and the information stored in the file employed to attain an end. Similarly if a unique identifier, e.g., a barcode is used, the unique identifier is usually read to identify, for example, an object or file associated with the unique identifier.

As used herein, the term “single nucleotide polymorphism”, or “SNP” for short, refers to single nucleotide position in a genomic sequence for which two or more alternative alleles are present at appreciable frequency (e.g., at least 1%) in a population.

The term “chromosomal region” or “chromosomal segment”, as used herein, denotes a contiguous length of nucleotides in a genome of an organism. A chromosomal region may be in the range of 20 nucleotides in length to an entire chromosome, e.g., 100 kb to 10 MB for example.

The term “sequence alteration”, as used herein, refers to a difference in nucleic acid sequence between a test sample and a reference sample that may vary over a range of 1 to 10 bases, 10 to 100 bases, 100 to 100 kb, or 100 kb to 10 MB. Sequence alteration may include single nucleotide polymorphism and genetic mutations relative to wild-type. In certain embodiments, sequence alteration results from one or more parts of a chromosome being rearranged within a single chromosome or between chromosomes relative to a reference. In certain cases, a sequence alteration may reflect an abnormality in chromosome structure, such as an inversion, a deletion, an insertion or a translocation, for example.

As used herein, the term “methyltransferase” refers to a family of enzymes that has an activity described as EC 2.11, according to the IUMBM enzyme nomenclature. In certain cases, methyltransferase catalyzes the transfer of a methyl group from a donor to an acceptor. The acceptor may be a nucleotide base in a DNA. When methylation occurs on a DNA, the methyltransferase is referred to as a DNA methyltransferase. DNA methyltransferases can use a cofactor such as s-adenosyl methionine (SAM) as the methyl donor in the methyltransferase reaction. In addition to methyl groups, methyltransferase may also transfer other functional groups to an acceptor from a cofactor, e.g. amino group, if used with an appropriate cofactor.

A “site-specific methyltransferase”, as used herein, denotes a methyltransferase that transfer either a methyl group, amino group, or another functional group to a site on a acceptor by recognizing specific regions on the acceptor. If the acceptor is a DNA molecule, the site-specific methyltransferase may recognize a specific sequence of nucleotide bases and transfers the functional group to a nucleotide base within the recognition sequence or close to the recognition sequence.

The term “s-adenosyl-methionine analog” or “SAM analog”, as used herein, denotes a cofactor that is a derivative of s-adenosyl-methionine. If a functional group replaces the methyl group attached to the sulfonium center, the SAM analog may act as a donor of the functional group instead of a donor of a methyl group.

As used herein, the term “data” refers to refers to a collection of organized information, generally derived from results of experiments in lab or in silico, other data available to one of skilled in the art, or a set of premises. Data may be in the form of numbers, words, annotations, or images, as measurements or observations of a set of variables.

The term “stretching”, as used herein, refers to the act of elongating a DNA molecule so to minimize the amount of tertiary structures, e.g. unfolding coiled DNA structures.

The term “homozygous” denotes a genetic condition in which identical alleles reside at the same loci on homologous chromosomes. In contrast, “heterozygous” denotes a genetic condition in which different alleles reside at the same loci on homologous chromosomes.

As used herein, the term “amino group-providing cofactor” refers to a compound required in catalytic reaction that involves tranferring an amino group to a substrate. An amino group-provided cofactor assists the enzyme by acting as a donor of a functional group comprising an amino group.

As used herein, the term “amine-reactive” describes a functional group that under certain conditions reacts with an amino group to form a covalent bond to the nitrogen of the amino group.

As used herein, the term “reactive amino group” denotes an amine that can react with another functional group to form a covalent bond between the nitrogen of the amino group and the electrophile of the functional group.

The term “amidated”, as used herein, refers to a biomolecule with an amino group covalently attached.

The term “methyltransferase reaction conditions”, as used herein, refers to conditions suitable for a methyltransferase to be active in transferring a functional group from a cofactor/donor to a substrate/acceptor.

The term “coding region”, as used herein, refers to a contiguous stretch of nucleotides (a nucleotide sequence) that provides the genetic information to encode a gene product (RNA and/or polypeptide). In contrast, “non-coding region” refers to nucleic acid sequences that do not encode a gene product, such as promoters, enhancers, centromeres, and telomere sequences, for example.

### DESCRIPTION OF EXEMPLARY EMBODIMENTS

A method of genome analysis is provided. In certain embodiments, the method may comprise: labeling the test genome using a first site-specific methyltransferase to produce a labeled test genome comprising a label; and analyzing the labeled test genome to determine if the test genome comprises a sequence alteration relative to a reference sequence.

Before the present invention is described in greater detail, it is to be understood that this invention is not limited to particular embodiments described, and as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Method of Genome Analysis

A method for analyzing a genome is provided. In certain embodiments, the method includes: labeling a test genome in a sample using a first site-specific methyltransferase (MTase) to produce a labeled test genome comprising a label; and analyzing the labeled test genome to determine if the test genome comprises a sequence alteration relative to a reference sequence. In certain embodiments, the method of analyzing the labeled test genome comprises: evaluating binding of the labeled test genome to an array of probes or observing a pattern of labeling along the labeled test genome.

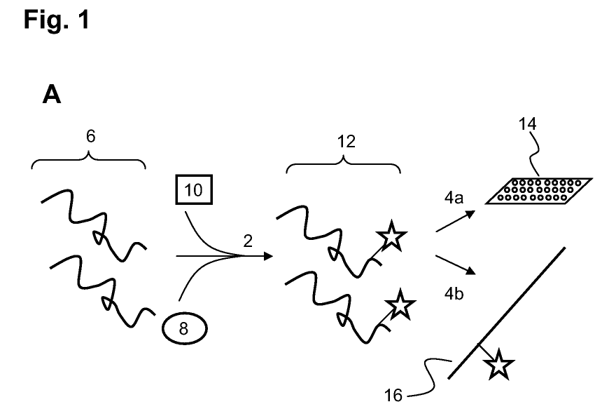

Certain features of the subject method are illustrated in FIG. 1 and are described in greater detail below. With reference to FIG. 1, the method involves contacting 2 a test genome 6 with a methyltransferase (MTase) 8 and a cofactor 10 under conditions suitable for the MTase to be active. The MTase then transfers a detectable label or a functional group reactive with a detectable label from the cofactor onto the test genome. The test genome becomes labeled 12 as a result of the labeling step 2 in a site-specific manner. The site-specific labels may be detected, e.g., using a microscope or an array, to provide test data. Since MTase-labeling is sequence dependent, the presence or absence of labeling in specific locations on the test genome is informative of the sequence information in those locations. By comparing the pattern and the intensity of the label signals of the labeled test genome to those of a reference sequence, the difference in sequence between the labeled test genome and the reference genome may be determined.

As shown in FIG. 1, the labeling step 2 may be performed by contacting the test genome 6 with the cofactor in the presence of an MTase under conditions that allow the transfer of a functional group from the cofactor to the genome. The cofactor may be introduced together with the MTase or combined with the test genome prior to the addition of MTase. The way and order of contacting the test genome and cofactor may vary depending on the assay conditions. In certain cases, the MTase may be added to a sample comprising the test genome. In other cases, the sample comprising the test genome may be added to a solution containing the MTase. Many other ways of contacting the genome, the cofactor, and the MTase may be employed. Conditions and reagents suitable for MTase reaction are known to one of skilled in the art. Exemplary methods and experimental conditions suitable for an active MTase may be found in Adams R L P et al. (“Microassay for DNA methyltransferase” J. Biochem. Biophys. Methods 22:19-22, 1991) and Yu Z et al. (“Hypermethylation of the inducible nitric-oxide synthase gene promoter inhibits its transcription” J. Biol. Chem. 279:469541-46961, 2004).

In one embodiment of a labeling reaction, the labeling step comprises a one-step transfer of a labeled functional group onto a genome. In this embodiment, the cofactor employed comprises a detectable label linked to the functional group. The cofactor with a detectable label already attached may be synthesized prior to the labeling step 2 and the details of making such a cofactor is known in the art, as exemplified by EP172557 and WO 2006/108678, disclosures of which are incorporated herein by reference. As such, the functional group transferred onto the genome by the MTase is a functional group already linked to a detectable label.

In an alternative embodiment, the labeling step may involve a two-step process in which the functional group to be transferred in the first step of this two-step process does not contain detectable label. In certain cases, the functional group may comprise a primary amine or a thiol, which the MTase transfers onto specific acceptor nucleotides of the genome. As a result of the first step, the genome is covalently modified to contain a first reactive functional group. In the case of a cofactor donating an amine as the first functional group, the genome is considered to be amidated. In the second step of this two-step process, a label comprising a second functional group that is known to be reactive with the first functional group is contacted with the modified genome produced by the first step. By “reactive,” it is meant that the second functional group would form a covalent bond with the first functional group under appropriate experimental conditions. Exemplary reactive pairs of functional groups will be discussed in more detail below and may also be found in EP172557 and WO 2006/108678, disclosures of which are incorporated herein by reference. One feature of the two-step process in the labeling step 2 is to allow a wide variety of different pairings of the two functional groups employed. Moreover, in contrast to the one-step process, the type of detectable labels may be chosen after the genome has been modified with the first functional group.

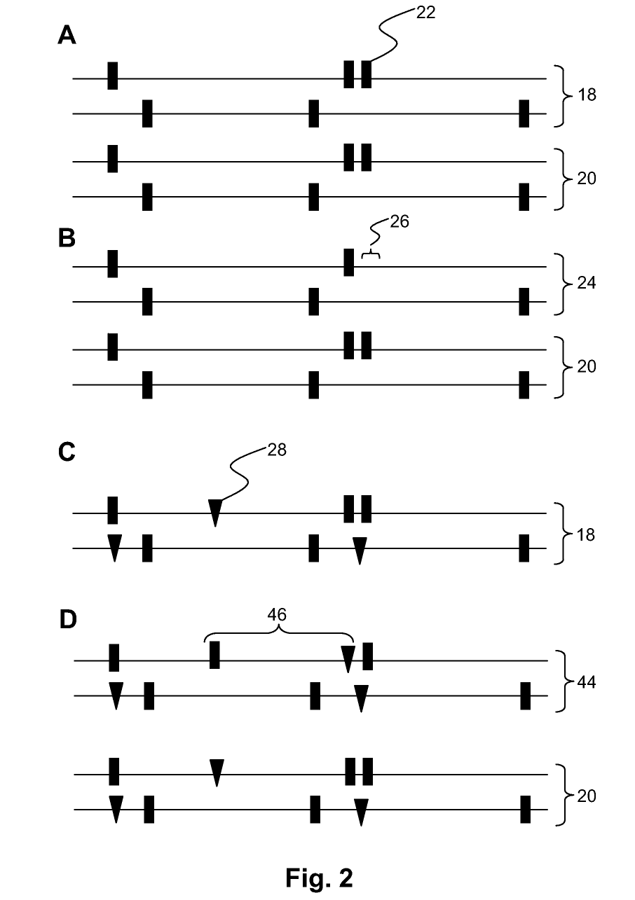

In certain embodiments, the method may employ more than one MTase, e.g. 2, 3, or more different types of MTases, in the labeling step. In certain cases, the multiple MTases may differ in the type of acceptor site to which they transfer the functional group. In other cases, the MTases may differ in the nucleic acid sequence that they recognize in the substrate genome. In the subject method where more than one MTase is employed, the labels each MTase incorporates onto the genome may be different in order to produce a labeled test genome with distinguishable patterns of labeling. For example, two different MTases may be used sequentially, each followed by coupling of two different fluorescent dyes. FIG. 2C illustrates certain features of an exemplary example that employs two MTases in the subject method in which the recognition sequences of the two MTases are different from each other. Solid rectangular bars 22 mark sites that are labeled by a first MTase and the solid triangles 28 denote sites that are labeled by a second MTase. As shown in FIG. 2C, using different labels that correspond to different MTase recognition sites, two patterns of labeling may be detected.

The labeling step may be carried out in vitro or in vivo. Cell extracts may be utilized in the labeling step. All steps of an in vitro labeling method may also be performed in a single tube. In other cases, the labeling step may be performed on a substrate. For example, the substrate genome may be immobilized onto a bead or a planar surface.

The MTase employed in the subject method refers to a family of enzymes that catalyze the transfer of a functional group (e.g. a methyl, amine, or thiol group) from one molecule as a donor to another as the acceptor. MTases may transfer the functional group to a protein, a nucleic acid, or other biomolecules. In certain embodiments, the site-specific MTase employed in the subject method is a DNA MTase. RNA methyltransferase may also be used in the subject method. The DNA MTase may be characterized by the acceptor site to which the functional group is transferred: C5 carbon of cytosine, N4 nitrogen of cytosine, or N6 nitrogen of adenine. In certain embodiments, the MTase is a site-specific MTase that recognizes a specific nucleotide sequence in the genome. In some cases, the recognition sequence may comprise 2, 3, 4, 5, 6, 8, 10 or more nucleotides or nucleotide pairs. Under suitable conditions, the site-specific DNA MTase specifically transfers a functional group from the cofactor to a nucleotide within or close to the recognition sequence. As such, in certain cases, the recognition sequence comprises an acceptor site for the functional group from the cofactor. As a result, the genome is modified by a covalent linkage to the functional group in a sequence- and base-specific manner. If the recognition sequence of the site-specific MTase does not exist in the genome, no functional group may be transferred from the cofactor to the genome.

In certain embodiments, the MTase may be a variant that exists in nature or a recombinant variant. Variants of MTase that may be used in the subject method include MTase protein variants or derivatives that are still enabled to transfer the functional group from a donor to an acceptor. It would be apparent to one of skilled in the art the variants of MTase that can be employed in the subject method since the structure and function relationships of MTase are known in the art, as illustrated in Chen X et al. 2008 “Mammalian DNA methyltransferases: A structural perspective” Cell 16:341-50.

The MTase may be of a bacterial restriction modification system or of a mammalian origin. In certain embodiments, bacterial MTases include but are not limited to M. Taq1, M. Hha1, M.Bcn1B, M.BseC1, M. Rsr1, M2.Bfi1, M2.Eco311. In certain cases, mammalian DNA MTases include but are not limited to DNMT1, DNMT2, DNMT3A and DNMT3B. Nucleotide and protein sequences of these exemplary bacterial or mammalian MTases are known and deposited in databases such as the NCBI's GenBank database.

As noted above, in certain embodiments, the DNA MTases modifies the genome in a sequence- and base-specific manner. In certain cases, the recognition sequence may comprise 2, 3, 4, 5, 6, 8, 10 or more nucleotides or nucleotide pairs. The acceptor nucleotide onto which the functional group is transferred may be within or close to the recognition site. For example, the bacterial HaeIII MTase recognizes 4 consecutive nucleotide bases of the sequence GGCC and transfers the functional group onto the internal cytosines (C5) of the recognition sequence. As an another example, the bacterial AluI MTase recognizes the panlindromic sequence of AGCT and transfers the functional group onto the internal cytosines (C5) of the recognition sequence. Many other MTases and the information relating to their recognition and acceptor sites are known in the art and commercially available.

In certain cases, the recognition sequence and/or the acceptor site of an MTase overlaps a site of single nucleotide polymorphism (SNP) in the test genome or reference sequence. Since the nucleotide sequences of hundreds of thousand of SNPs from humans, other mammals (e.g., mice), and a variety of different plants (e.g., corn, rice and soybean), are known (see, e.g., Riva et al 2004, A SNP-centric database for the investigation of the human genome BMC Bioinformatics 5:33; McCarthy et al 2000 The use of single-nucleotide polymorphism maps in pharmacogenomics Nat Biotechnology 18:505-8) and are available in public databases (e.g., NCBI's online dbSNP database, and the online database of the International HapMap Project; see also Teufel et al 2006 Current bioinformatics tools in genomic biomedical research Int. J. Mol. Med. 17:967-73), the labeling of genomic DNA using an MTase to identify an SNP is well within the skill of one of skilled in the art. The SNP may be known prior to choosing an MTase based on the MTase recognition site. In certain embodiments, individual SNPs may destroy certain MTase recognition sequences that are present in the human genome reference sequence, and other SNPs may create MTase recognition sequences that are not present in the human genome reference sequence. Therefore, individual DNA samples may have different patterns of MTase recognition sequences resulting from SNP variations. For example, a SNP that lies within a sequence comprising the recognition sequence of bacterial AluI MTase may cause the sequence to be AGCT in certain individuals and CGCT in other individuals. As a result, the pattern of labeling of a test genome may be different from that of a reference genome depending on the SNP that resides in those recognition sequences.

Another component that is employed in the labeling step of the subject method is a donor, which is also referred to as a coenzyme or a cofactor. In certain embodiments, the cofactor is a derivative of S-adenosyl-L-methionine (SAM or AdoMet). In nature, DNA MTase catalyzes the nucleophilic attack of the amino group of a nucleotide base onto a methyl group of SAM to result in the methyl group transfer. In certain embodiments of the subject method, a SAM analog is used as the cofactor such that other chemical moieties or functional group may be transferred onto the genome other than a methyl group. In certain cases, the SAM analog contains a double bond, triple bond, aromatic, or heteroaromatic moiety in the β-position to the sulfonium center. Examples of substituents of the sulfonium center are allylic, propargylic, and benzylic substituents. Exemplary SAM analog may have prop-2-enyl (—CH₂CH═CH₂), prop-2-ynyl (—CH₂C≡CH), but-2-ynyl (—CH₂C≡CCH₃), pent-2-ynyl (—CH₂C≡CCH₂CH₃), or benzyl group at the sulfonium center. The functional group, such as a primary amine or a thiol to be transferred onto the genome may be attached to the exemplary allylic system via a spacer. More details on the various SAM analog that may be used in the subject method can be found in EP172557 and WO 2006/108678, disclosures of which are incorporated herein by reference.

As noted above, a label used in the subject method may be attached to the functional group on the SAM analog prior to the labeling step to be used in a one-step labeling method. In another embodiment, the label may be subsequently attached after the transfer of the first functional group onto an acceptor nucleotide of the test genome. In the latter method that involves a two-step process, a label that is reactive with the first functional group is provided, so that a covalent linkage may be formed between the label and the functional group on the genome.

In an embodiment where the SAM analog comprises a first functional group with an amine, the SAM analog may act as an amine-providing cofactor. The MTase transfers the functional group with the amine to an acceptor nucleotide on the genome. In order to be covalently bonded to the first functional group on the genome, the label to be attached onto the genome comprises an amine-reactive functional group. Exemplary amine-reactive functional groups include but are not limited to N-hydroxylsuccinimidyl ester, acyl azide, acyl nitrile, acyl chloride, pentafluorophenyl ester, thioester, sulfonyl chloride, isothiocyanate, imidoester, aldehyde, and ketone. The primary amine of the functional group on the test genome reacts with the amine-reactive groups to form a covalent linkage, e.g. amide bond. In an embodiment where the first functional group is a thiol, the label may comprise a thiol-reactive functional group. Exemplary thiol-reactive functional groups include but are not limited to haloacetamide, maleimide, aziridine, and another thiol. The thiol group as the functional group on the test genome reacts with the thiol-reactive groups on the labels to from a covalent bond, e.g. thioester, or disulfide. Many other pairs of reactive groups may be employed such that the functional group transferred from the cofactor onto the test genome may be covalently linked to a detectable label. More details on the reactive groups may be found in EP172557 and WO 2006/108678, disclosures of which are incorporated herein by reference.

In addition to having a functional group that is reactive for the formation of a covalent bond to the first functional group transferred onto the test genome, the label also comprises a detectable component that is subsequently used for analysis. Detectable labels are known in the art and need not described in detail herein. Briefly, exemplary detectable components include radioactive isotopes, fluorophores, fluorescence quenchers, affinity tags, e.g. biotin, crosslinking agents, chromophores, beads, etc. In certain embodiments, the detectable label, such as a fluorophore, may be detected directly without performing additional steps. In other embodiments, the detectable label, such as biotin, may require incubation with a recognition element, such as streptavidin, or with secondary antibodies to yield detectable signals.

As mentioned above, the subject method comprises analyzing the labeled test genome 4, e.g., using an array 4a or by stretching out the labeled test genome 4b, to provide test data. In certain embodiments, the analyzing step 4 involves detecting the label on the labeled test genome 12 obtained from the labeling step 2. If the label is fluorescent, the presence of the label may be detected by the human eye, a camera, flow cytometry, or other fluorescence detectors, such as a spectrometer. If the label is a tag composed of synthetic compounds, nucleic acids, amino acids, or a combination of both nucleic acids and amino acids, the tag may be detected via binding to an epitope presented on the tag, primer extensions, sequencing, or additional processing to identify and locate the label, for example.

In certain cases, the labeled genome is stretched out into a linear or close to linear form in order to detect the labels on the genome. Double-stranded DNA in aqueous solutions usually assumes a random-coil conformation. Similar to the method used in Fiber-FISH, the labeled genome comprising coiled DNA molecules may be unwound and stretched into a linear form on a modified glass surface and individually imaged by microscopy, e.g. confocal, epifluorescence, internal reflection fluorescence. Briefly, the method may involve the following steps. First, the genome is pipetted onto the edge of a glass slide. The solution of the genome is then drawn under the coverslip by capillary action, causing the DNA molecules of the genome to be stretched and aligned on the coverslip surface. As a result, an array of combed single DNA molecules is prepared by stretching molecules attached by their extremities to a glass surface with a receding air-water meniscus. This method is also referred to as molecular combing. By detecting the labels on the combed DNA, label position in the context of the whole chromosomal segment may be directly visualized, providing a means to construct physical maps and to detect micro-rearrangements. Details of a method using microscopy to detect stretched genomic DNA may be found in Xiao M et al. (2007) “Rapid DNA Mapping by fluorescent single molecule detection” Nucleic Acids Res. 35:e16.

In other embodiments, the DNA molecules of the genome may be stretched as they flow through a microfluidic channel. The hydrodynamic forces in a microfluidic channel generated in laminar flow help to uncoil and to stretch the DNA molecules as they travel with the flow. The solution is pressure driven to provide a flow acceleration over a distance comparable to the size of the DNA molecule. In this approach, a stretched DNA molecule travels through posts of focused light to excite a fluorophore label, for example. The label is detected as the DNA molecules pass through the detectors placed appropriately to capture the signal emitting from the microchannel. Details of using microfluidic channel to stretch and analyze single molecules may be found in US Pat Pub 20080239304 and 20080213912, disclosures of which are incorporated herein by reference.

In alternative embodiments, the DNA molecules of the genome may be stretched as they flow through a nanofluidic channel. In these embodiments, the nanofluidic channel may have a diameter of less than 200 nm, for example, less than 150 nm, less than 100 nm, less than 50 nm, or less than 20 nm. The confinement of the DNA molecules in the nanochannels leads to elongation of the DNA molecules, allowing optical interrogation. See e.g., Tegenfeldt et al (2004) “The dynamics of genomic-length DNA molecules in 100-nm channels” Proc. Nat. Acad. Sci. USA 101:10979-10983.

In certain embodiments, the labeled test genome is hybridized to an array containing probes designed to detect regions on the genome comprising MTase recognition sites and/or acceptor sites. The probes on the array may be complementary to regions of the genome predicted to be labeled by the MTase. In other embodiments, the probes may be complementary to regions of the genome predicted to be recognized by the MTase. The test genome may be amplified, purified, fragmented, or further processed prior to hybridization to the array. Hybridization to an array is usually followed by washing steps and detection of labeled nucleic acids bound to probes on the array. Details of methods involving array hybridization are known in the art. In certain embodiments, presence of a labeled hybridized target to the probe is an indication of the presence of an MTase recognition sequence in the genome. In other cases, an absence of such label indicates the lack of such an MTase recognition sequence.

The subject arrays may contain features in single sets, in pairs or in a plurality of sets, in which each set, pair, or plurality detects a single SNP or a single recognition site of an MTase. In certain cases, the array may contain only one feature or one type of oligonucleotide probe for detecting each SNP. In certain embodiments, each subject array may contain more than one such feature, and those features may correspond to (i.e., may be used to detect) a plurality of SNPs and/or MTase recognition sites. Accordingly, the subject arrays may contain a plurality of features (i.e., 2 or more, about 5 or more, about 10 or more, about 15 or more, about 20 or more, about 30 or more, about 50 or more, about 100 or more, about 200 or more, about 500 or more, about 1000 or more, usually up to about 10,000 or about 20,000 or more features, etc.), each containing a different corresponding sequence to detect different SNPs and/or MTase recognition sites. In certain embodiments, therefore, the subject arrays contain a plurality of oligonucleotide features that correspond to a plurality of SNPs and/or MTase recognition sites of a genome. In particular embodiments, therefore, the subject arrays may contain features to detect, i.e., corresponding to, all of the predicted SNPs of a particular genome. The subject arrays may contain at least up to at least 45,000 different features to detect SNPs and MTase recognition sites.

In general, arrays suitable for use in performing the subject method contain a plurality (i.e., at least about 100, at least about 500, at least about 1000, at least about 2000, at least about 5000, at least about 10,000, at least about 20,000, usually up to about 100,000 or more) of spatially addressable features containing oligonucleotides that are linked to a usually planar solid support. In an alternative embodiment, an array suitable for performing the subject method may be optically addressed. The probes of the array may be linked to beads, each containing a unique optical signature that may be detected and decoded.

In particular embodiments, SNPs or MTase recognition sites of interest may be detected by 1, 2, about 5, or about 10 or more, e.g., up to about 20 sets of surface-tethered oligonucleotide features. Such an array may contain duplicate oligonucleotides or different surface-tethered oligonucleotides for the same SNP or MTase recognition site.

In general, methods for the preparation of polynucleotide arrays are well known in the art (see, e.g., Harrington et al, Curr. Opin. Microbiol. (2000) 3:285-91, and Lipshutz et al., Nat. Genet. (1999) 21:20-4) and need not be described in any great detail. The subject oligonucleotide arrays can be fabricated using any means, including drop deposition from pulse jets or from fluid-filled tips, etc, or using photolithographic means. Either polynucleotide precursor units (such as nucleotide monomers), in the case of in situ fabrication, or previously synthesized polynucleotides can be deposited. In some embodiments, the arrays may be constructed to include oligonucleotide analogs such as nucleotide analogs such as 2,6-aminopurines. Such methods are described in detail in, for example U.S. Pat. Nos. 6,242,266, 6,232,072, 6,180,351, 6,171,797, 6,323,043, and U.S. Patent Application US20040086880 A1, etc., the disclosures of which are herein incorporated by reference.

As noted above, the subject method comprises analyzing the labeled test genome to provide test data. Depending on the specific embodiment of the analyzing step 4, different format of test data may be obtained. If fluorescence detection is carried out on a stretched fluorescently labeled DNA molecule, the test data may comprise information indicating the presence or absence of fluorescence on specific locations of a DNA molecule. In certain cases, the test data record more than one labeling pattern from DNA molecules that have more than one type of fluorescent label (e.g., FIG. 2C). In certain embodiments, the data incorporate information derived from DNA molecules labeled with a nonspecific label, such as an intercalating fluorescent dye. In certain cases, a pattern of fluorescent labels may be recorded in forms of images or tables correlating the signal intensity over chromosomal length. If an array-based experiment is performed to provide the test data, the test data may be presented as values of signal intensities at each feature location. The feature location may be identified by the probe sequence or the region of the genome to which the probe is designed to hybridize.

The subject method also comprises analyzing the test data to determine if the test genome comprises a sequence alteration relative to a reference sequence. In certain embodiments, the sequence alterations that may be detected include translocations, inversions, tandem duplications, insertions, deletions, SNPs, and other sequence mutations. An exemplary case is illustrated in FIGS. 2A and 2B, in which test genomes 18 and 24 are compared to the reference sequence 20. The parallel lines represent two alleles (top and bottom) for each genome presented, with solid rectangles representing sites labeled by an MTase. In FIG. 2A, both the top and bottom alleles of test genome 18 have the same pattern of labeling as the alleles of the reference sequence 20. In contrast, if the test genome has a mutation at the recognition site and/or acceptor site of the MTase, the labeling pattern would be different from a reference sequence without the mutation. Certain features of sequence analysis are illustrated in FIG. 2B, in which genome 24 has a mutation at site 26 on the top allele. Relative to the reference sequence 20, the top allele is missing one label at site 26. A second exemplary case is illustrated in FIG. 2D, in which test genome 44 is compared to the reference sequence 20. In FIG. 2D, the pattern of labels in the top allele of genome 44 is consistent with a chromosomal inversion at site 46. By detecting and comparing labeling patterns in ways analogous to the above embodiments, chromosomal abnormalities, SNP, and other genetic variations may be determined relative to a reference sequence.

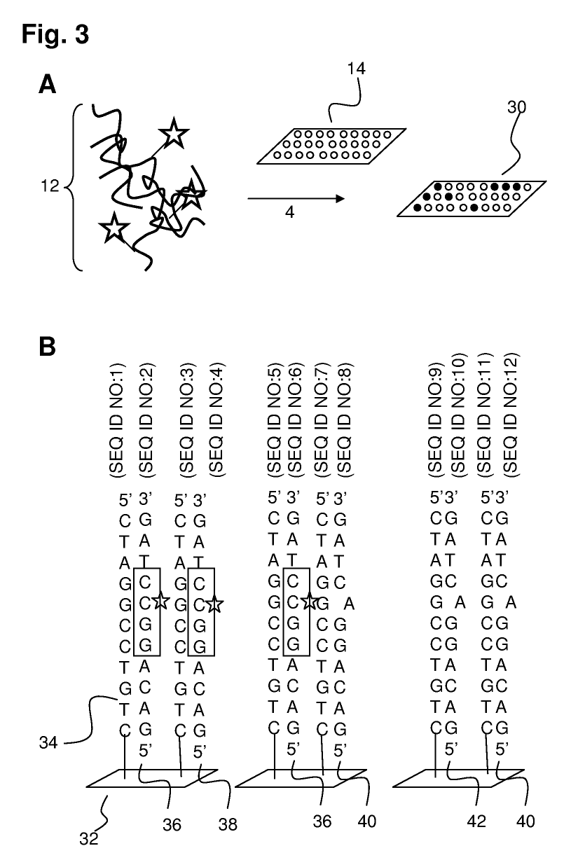

In other embodiments, the sequence alterations may be detected by an array-based assay, as described above. The sequence alterations may include SNPs that give rise to a loss of heterozygosity (LOH). Recognition sequences of certain MTases overlap sites for SNPs. The SNP may be linked to a phenotype (e.g., a disease) or may be unlinked to a phenotype (e.g., may be an “anonymous” SNP). Depending on the nucleotide at the SNP site, MTase may or may not recognize the nucleotide sequence to catalyze the labeling reaction. Certain features of the array analysis are illustrated in FIG. 3. With reference to FIG. 3A, a test genome is labeled and hybridized to an array 14, where certain probes are hybridized to targets that emit detectable signals. In the exemplary embodiment shown in FIG. 3B, if the test genome is homozygous for the SNP recognizable by the MTase used, both alleles would be labeled (allele 36 and 38). If the test genome is heterozygous (allele 36 and 40 in FIG. 3B), in which one allele (40) possesses an SNP not recognizable by the MTase used, then one allele would not be labeled. Consequently, the detectable signal would be approximately one half of the signals of the genome that is homozygous (36 and 38). In a situation where both alleles (40 and 42) of a test genome lose the SNP sites recognizable by the MTase used, there would be no signal beyond background, relative to the genome homozygous for the recognition site. This method of comparing the amount of signals in a test genome relative to a reference may be used to detect LOH.

In carrying out the analysis using test data, a reference sequence may be used in certain embodiments. A reference sequence may be a sequence derived from an identified source. The source may be known to be homozygous or heterozygous for a particular genomic locus of interest. In certain cases, the source may be wild-type for a genomic locus of interest. The source may contain an allelic variant of interest. In certain cases, the reference sequence may be known so that the specific nucleotide sequences implicated in single nucleotide polymorphism, restriction fragment length polymorphism, genetic mutations, etc, are known. The reference sequence may also undergo the subject method so that it is labeled using an MTase to provide reference data. In other embodiments, the reference data may be derived in silico based on the information available about the reference sequence, such as those stored in databases. For example, the pattern of labeling may be predicted based on sequence data and the recognition site of the MTases used.

In certain cases, the structural overview of the test genome may be provided by analyzing the labeling patterns of a specific MTase. The structures that may be identified include but not limited to AT-rich repeats, telomeres, and centromeric sequences. Since some site-specific MTase have recognition sequences that consist of exclusively guanines and/or cytosines, e.g. HaeIII, a high density of labels by such MTases along a stretch of test genome indicates a region with a high content of guanines and cytosines. Regions with a high content of guanines and cytosines are likely to be coding regions or a random sequence. As such, AT-rich repeats, telomeres, and centromeric sequences would be labeled at a lower density than coding regions or a random sequence. Comparing the density of MTase labeling between different regions of a test genome then allows for structural mapping of the test genome.

### Kits

Also provided by the subject invention are kits for practicing the subject method, as described above. The subject kit contains a site-specific MTase, a methyltransferase cofactor, reagents for labeling a test genome. The kit may further contain a reference genome or information relating to a reference genome.

In additional embodiments, the kit may further comprise an array of probes that are complementary to nucleic acid sequences recognized by the MTase. In an alternative embodiment, the kit further comprises an array of probes predicted to be complementary to chromosomal segments comprising sites recognized by the enclosed MTase.

The kits may be identified by the type of site-specific MTase, the recognition sequence of the MTase, the acceptor site of the MTase, or the reference genome. The kits may also be identified by the type of cofactor in the kit, e.g. a specific type of SAM analog and the type of functional group or chemical moiety linked to the SAM analog. The kits may be further identified by the method of analyzing the labeled genome.

In addition to above-mentioned components, the subject kit typically further includes instructions for using the components of the kit to practice the subject method. The instructions for practicing the subject method are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

In addition to the instructions, the kits may also include one or more control analyte mixtures, e.g., two or more control analytes for use in testing the kit.

### Utility

The subject method finds use in a variety of applications, where such applications are generally nucleic acid detection applications in which the presence of a particular nucleotide sequence in a given sample is detected at least qualitatively, if not quantitatively. In general, any assay involving the use of an MTase to identify the presence of a nucleotide sequence in a genome is provided by the subject method.

Specific genome analysis applications of interest include but are not limited to SNP detection assays. One embodiment of SNP detection assays employs an array-based method to detect loss of heterozygosity. In this embodiment, the amount of signal emitted by the labels depends on the presence of specific recognition sequences on the test genome containing the SNP, as discussed above. The amount of signal is then compared to the amount of signal from a reference sequence. If the amount of signal is the same between the test genome and the reference genome, then the SNP of the test genome is the same as that of the reference. For example, if both alleles of the reference genome are labeled by an MTase, the sequence is homozygous for a SNP that allows labeling by the MTase. If the amount of signal in the reference genome differs by half, then one of the two alleles (in a diploid organism) is different from the reference sequence, possibly indicating heterozygosity. If there is no signal above background levels in the test genome, the sequence is likely to be homozygous for a SNP which does not allow labeling by the MTase. If a plurality of alleles in a given chromosomal region are found to be homozygous, there may be a loss of heterozygosity in that region. Statistical methods for predicting LOH are known in the art. See Beroukhim et al, PLoS Comput Biol. (2006) 2:e41, for example.

In certain cases, the test genome may be derived from a sample tissue suspected of a disease or infection. Performing the subject method to analyze the test genome from such sample tissues would be useful for disease diagnosis and prognosis. Patents and patent applications describing methods of using arrays in various applications include: U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference.

Another application of interest may be to carry out genome analysis on a single molecule level, using methods such as those involving microscopy or a microfluidic channel. In particular embodiments, the test genome or regions of interest are subjected to DNA stretching or confinement elongation to ease analysis. Comparing the labeling pattern resulted from MTase labeling on a chromosomal segment with a reference sequence may identify genetic mutations and chromosomal rearrangements, such as inversion, translocation, deletion, or duplications. Other assays of interest which may be practiced using the subject method include: genotyping, scanning of known and unknown mutations, gene discovery assays, genomic structural mapping, differential gene expression analysis assays, nucleic acid sequencing assays, and the like.

The above described applications are merely representations of the numerous different applications for which the subject array and method of use are suited. In certain embodiments, the subject method includes a step of transmitting data from at least one of the detecting and deriving steps, as described above, to a remote location. By “remote location” is meant a location other than the location at which the array is present and hybridization occur. For example, a remote location could be another location (e.g., office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart. “Communicating” information means transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, internet, etc.

In certain embodiments of the subject methods in an array, the array may typically be read. Reading of the array may be accomplished by illuminating the array and reading the location and intensity of resulting fluorescence at each feature of the array to detect any binding complexes on the surface of the array. For example, a scanner may be used for this purpose which is similar to the AGILENT MICROARRAY SCANNER device available from Agilent Technologies, Santa Clara, Calif. Other suitable apparatus and methods are described in U.S. Pat. Nos. 5,091,652; 5,260,578; 5,296,700; 5,324,633; 5,585,639; 5,760,951; 5,763,870; 6,084,991; 6,222,664; 6,284,465; 6,371,370 6,320,196 and 6,355,934; the disclosures of which are herein incorporated by reference. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere). Results from the reading may be raw results (such as fluorescence intensity readings for each feature in one or more color channels) or may be processed results such as obtained by rejecting a reading for a feature which is below a predetermined threshold and/or forming conclusions based on the pattern read from the array (such as whether or not a particular target sequence may have been present in the sample). The results of the reading (processed or not) may be forwarded (such as by communication) to a remote location if desired, and received there for further use (such as further processing).

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. A method of analyzing a test mammalian genome comprising:
a) labeling a nucleic acid of said test mammalian genome using a first site-specific methyltransferase to produce a labeled nucleic acid comprising a label;
b) analyzing said labeled nucleic acid test to determine if said test mammalian genome comprises a chromosomal rearrangement or a different allele of a SNP relative to a reference mammalian sequence.

2. The method of claim 1, wherein said analyzing said labeled nucleic acid comprises:
a) evaluating binding of said labeled nucleic acid to an array of probes; or
b) observing a pattern of labeling along said labeled nucleic acid.

3. The method of claim 1, wherein said site-specific methyltransferase recognizes a site that comprises a SNP nucleotide.

4. The method of claim 3, wherein only one allele of said SNP is labeled by said site-specific methyltransferase.

5. The method of claim 4, wherein said analyzing comprises inferring the allele of said SNP from the labeling of said site.

6. The method of claim 2, wherein said probes are complementary to chromosomal segments comprising sites recognized by said site-specific methyltransferase.

7. The method of claim 2, wherein said analyzing comprises
a) hybridizing said labeled nucleic acid to an array;
b) detecting binding of said labeled nucleic acid to said array to provide test data; and
c) comparing said test data to reference data.

8. The method of claim 2, wherein said evaluating comprises stretching or elongating said labeled nucleic acid.

9. The method of claim 8, wherein said stretching comprises using a fluidic channel.

10. The method of claim 8, wherein said stretching comprises stretching said labeled nucleic acid on a substrate.

11. The method of claim 1, wherein said method comprises:
a) labeling a reference genome with said first site-specific methyltransferase to produce a second labeled genome; and
b) analyzing said second labeled genome to produce reference data;
c) comparing said test data to said reference data to determine a sequence alteration between said test data and said reference data.

12. The method of claim 1, wherein said reference sequence is a known sequence.

13. The method of claim 1, wherein said labeling said nucleic acid comprises:
a) combining said nucleic acid with a site-specific methyltransferase in the presence of an amino group-providing cofactor under methyltransferase reaction conditions, to produce an aminylated test genome comprising a reactive amino group; and
b) reacting an amine-reactive label with said reactive amino group to produce said labeled nucleic acid.

14. The method of claim 1, wherein said labeling said test genome comprises:
combining said test genome with a site-specific methyltransferase in the presence of a cofactor for said methyltransferase under methyltransferase reaction conditions, wherein said cofactor comprises a label and said methyltransferase transfers said label onto said test genome to produce said labeled test genome.

15. The method of claim 1, wherein said labeling comprises:
a) labeling said test genome using a second site-specific methyltransferase to produce a labeled test genome labeled with two different labels.

16. The method of claim 1, wherein said label is a fluorescent label.

17. The method of claim 1, wherein said labeling comprises combining a nucleic acid of said test mammalian genome with a site-specific methyltransferase that recognizes a nucleic acid sequence that is present at a higher density in coding regions of said genome than in non-coding regions.

18. The method of claim 13, wherein said cofactor is an s-adenosyl-methionine analog.

19. A kit for analyzing a test genome according to the method of claim 1 comprising:
a) a site-specific methyltransferase;
b) a methyltransferase cofactor;
c) reagents for labeling a test genome;
d) reference genome; and
e) instructions for performing the method of claim 1.

20. The kit of claim 19, wherein said kit further comprises an array comprising probes complementary to chromosomal segments comprising sites recognized by said methyltransferase.
